(19) United States
(12) Patent Application Publication
(10) Pub. No.: US 2003/0009213 A1
(43) Pub. Date: Jan. 9, 2003

(54) Stent having cover with drug delivery capability

(76) Inventor: Jun Yang, Dove Canyon, CA (US)

Correspondence Address: Raymond Sun, Law Offices of Raymond Sun, 12420 Woodhall Way, Tustin, CA, 92782, US

(21) Appl. No.: 10/236,611

(22) Filed: Sep. 5, 2002

Related U.S. Application Data
(63) Continuation-in-part of application No. 09/524,244, filed on Mar. 13, 2000.
(60) Provisional application No. 60/317,354, filed on Sep. 5, 2001.

(51) Int. Cl. A61F 2/06

(52) U.S. Cl. 623/1.13; 623/1.42

(57) ABSTRACT

A prosthesis has a tubular stent and a cover provided about the outer periphery of the stent. The cover can be made from a water absorbent material, and/or a matrix of protein. The present invention also provides a method for treating a vulnerable plaque, the method including implanting the prosthesis over the vulnerable plaque.

### BACKGROUND OF THE INVENTION

1. Related Cases

This is a continuation-in-part of co-pending Ser. No. 09/524,244, filed Mar. 13, 2000, entitled “Stent Having Cover With Drug Delivery Capability”, the entire disclosure of which is incorporated by this reference as though set forth fully herein. This also claims priority from Provisional Application No. 60/317,354, filed Sep. 5, 2001, entitled “Stent Having Cover With Drug Delivery Capability”.

2. Field of the Invention

The present invention relates to prostheses for implantation into a mammalian vessel, and in particular, to intraluminal stents that are provided with a cover that can deliver and release drugs.

3. Description of the Prior Art

The treatment of stenosis is the subject of much research and discussion. Stenosis is currently being treated by a number of well-known procedures, including balloon dilatation, stenting, ablation, atherectomy or laser treatment.

Restenosis is the renarrowing of a peripheral or coronary artery after trauma to that artery caused by efforts to open a stenosed portion of the artery, such as by balloon dilatation, ablation, atherectomy or laser treatment of the artery. For such procedures, restenosis occurs at a rate of about 20-50% depending on the definition, vessel location, lesion length and a number of other morphological and clinical variables. Restenosis is believed to be a natural healing reaction to the injury of the arterial wall that is caused by angioplasty procedures. The host reaction begins with the thrombotic mechanism at the site of the injury. The final result of the complex steps of the healing process can be intimal hyperplasia, the uncontrolled migration and proliferation of medial smooth muscle cells, combined with their extracellular matrix production, until the artery is again stenosed or occluded.

Many attempts have been made or suggested to treat stenosis, and to prevent or minimize restenosis. One common approach is to implant intravascular stents in coronary and peripheral vessels. The stent is usually inserted by a delivery system (e.g., such as a catheter) into a vascular lumen and expanded (either via a balloon on a catheter, or through self-expansion) into contact with the diseased portion of the arterial wall to provide mechanical support for the lumen. The positioning of the stent in the lumen can be used to treat stenosis by re-opening the lumen that had been partially blocked by the stenosis. However, it has been found that restenosis can still occur with such stents in place. In addition, a stent itself can cause undesirable local thrombosis. To address the problem of thrombosis, persons receiving stents also receive extensive systemic treatment with anti-coagulant and antiplatelet drugs.

To address the restenosis problem, a number of approaches have been suggested. One type of approach relates to the delivery of drugs to minimize restenosis. As one example, these drugs can be delivered via oral, intravascular or intramuscular introduction, but these attempts have been largely unsuccessful. Unfortunately, pills and injections are known to be ineffective modes of administration because constant drug delivery and higher local concentration are very difficult to achieve via these means. Through repeated doses, these drugs often cycle through concentration peaks and valleys, resulting in time periods of toxicity and ineffectiveness.

Localized drug delivery is another example. There were many different attempts to provide localized drug delivery. One example of localized drug delivery is to provide the metallic walls or wires of the stents with therapeutic substances, fibrin and other drugs that can be released over a period of time at the diseased location of the vessel. However, the incorporation of drug into the walls or wires of the stent may significantly compromise the strength of the stent.

A second example of localized drug delivery is to incorporate a drug into a stent that is constructed not of metal but of a biodegradable polymer. However, the loading in and releasing of drugs from a polymeric stent may change the structural integrity and mechanical properties of the stent.

A third example of localized drug delivery is to directly coat the metal stent with a polymer that is bonded to or contains the desired drugs or anti-stenotic substances. Unfortunately, such polymer-coated stents have not been completely effective in preventing restenosis because of the cracking of the polymer as the stent is being expanded during deployment, saturation of the drug binding sites on the stent, and other reasons.

A fourth example of localized drug delivery is to provide a polymer sleeve or sheath that encompasses a portion of the stent. The sleeve or sheath would operate as a local drug delivery device. In some instances, the sheath or sleeve is made up of a bioabsorbable polymer that incorporates a drug, with the sheath or sleeve having a thickness to allow for controlled release of the drug. However, this approach suffers from the drawback that very few drugs are capable of being incorporated with common solid state polymers. In addition, directional release of drug to either the lumen or the arterial wall cannot be achieved. It will also be problematic for medical practitioners to select the type of drug and the dosage of the drug to be used, as well as the stent type to be implanted.

In addition to the problems of stenosis and restenosis, the development of cancerous blockages inside body passageways (e.g., esophagus, bile ducts, trachea, intestine, vasculature and urethra, among others) can also be treated with stents, which operate to hold open passageways which have been blocked by the cancerous growth or tumors. However, the stents do not prevent the ingrowth of the cancerous material through the interstices of the stent. If the ingrowth reaches the inside of the stent, it might result in blockage of the body passageway in which the stent had been implanted.

In addition to the above-described problems experienced by localized drug delivery, conventional stents are also ineffective in preventing the ingrowth of host tissue proliferation or inflammatory material through the interstices of the stent. Some inflammatory reactions may be associated with vulnerable plaque or other unknown causes.

Further, traditional scientific wisdom holds that heart attacks originate from severe blockages created by atherosclerosis (i.e., the progressive build-up of plaque in the coronary arteries). The increase of lipids in the artery and the ensuing tissue reaction lead to narrowing of the affected vessel which, in turn, can result in angina and eventual coronary occlusion, sudden cardiac death, and thrombotic stroke. However, research conducted in the past decade is leading to a shift in understanding of atherosclerosis and pointing the way to major changes in the diagnosis and treatment of some kinds of life threatening forms of heart disease.

Scientists theorize that at least some coronary diseases are inflammatory processes, in which inflammation causes plaque to rupture. These so-called “vulnerable plaques” do not block the arteries. On the other hand, much like an abscess, they are ingrained under the arterial wall, so that they are undetectable. They cannot be seen by conventional angiography or fluoroscopy and they do not cause symptoms such as shortness of breath or pain. Yet, for a variety of reasons, they are more likely to erode or rupture, creating a raw tissue surface that forms scars. Thus, they are more dangerous than other plaques that cause pain, and may be responsible for as much as 60-80% of all heart attacks.

As used herein, the term “restenosis” is defined to be a natural healing reaction to the injury of the arterial wall that is caused by angioplasty procedures. “Restenosis” is not associated with vulnerable plaque. The host reaction begins with the thrombotic mechanism at the site of the injury. The final result of the complex steps of the healing process can be intimal hyperplasia, the uncontrolled migration and proliferation of medial smooth muscle cells, combined with their extracellular matrix production, until the artery is again stenosed or occluded as typically observed in a stable plaque as opposed to a vulnerable plaque.

Thus, there still remains a need for a prosthesis that provides effective localized drug delivery to minimize or prevent restenosis and the ingrowth of host tissue proliferation or inflammatory material through the interstices of the stent, while avoiding the disadvantages set forth above. There also remains a clinical need for a method for treating vulnerable plaque.

### SUMMARY OF THE DISCLOSURE

It is an object of the present invention to provide an intraluminal prosthesis that minimizes or prevents the ingrowth of host tissue proliferation or inflammatory material through the interstices or ends of a stent.

It is another object of the present invention to provide an intraluminal prosthesis that provides effective localized drug delivery.

It is yet another object of the present invention to provide an intraluminal prosthesis that provides site-specific drug delivery and/or evenly distributed drug delivery for treating a region of the intraluminal surface.

It is yet a further object of the present invention to provide a method for treating vulnerable plaque.

In order to accomplish the objects of the present invention, there is provided a prosthesis having a tubular stent and a cover provided about the outer periphery of the stent. The cover can be made from a water absorbent material, and/or a matrix of protein. In one embodiment of the present invention, the cover is made from either tissue or hydrogel.

The present invention also provides a method for treating a vulnerable plaque, the method including implanting a prosthesis over the vulnerable plaque, wherein the prosthesis has a stent and a cover surrounding at least a portion of both an inner periphery and an outer periphery of the stent.

### BRIEF DESCRIPTION OF THE DRAWINGS

FIG. 1 is a schematic view of an intraluminal prosthesis according to one embodiment of the present invention.

FIG. 1A illustrates one method of attaching the cover to the stent of the prosthesis of FIG. 1.

FIG. 1B illustrates another method of attaching the cover to the stent of the prosthesis of FIG. 1.

FIG. 2A is another schematic view of the prosthesis of FIG. 1.

FIG. 2B is a cross-sectional view of the prosthesis of FIG. 2A.

FIG. 3 illustrates yet another method of attaching the cover to the stent of the prosthesis of FIG. 1.

FIG. 4 is a cross-sectional view of the prosthesis of FIG. 3.

FIG. 5 is a schematic view of a prosthesis according to the present invention that utilizes a patch.

FIG. 5A illustrates a portion of the prosthesis of FIG. 5 when the prosthesis is in the collapsed configuration.

FIG. 5B illustrates a portion of the prosthesis of FIG. 5 when the prosthesis is in the expanded configuration.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention provides an intraluminal prosthesis that has an underlying stent with a cover acting as a sheath or sleeve. The cover acts as a drug delivery device for locally delivering a drug to a vessel wall or lumen into which the prosthesis has been inserted and positioned. The cover may also function to block the path of cell migration (i.e., ingrowth), and to pave or act as a scaffold for supporting the lumen, such as in stenosis, restenosis, tumorous, or vulnerable plaque treatment.

The cover of the present invention can be provided in the form of a patch.

The tubular cover or patch cover can be generally made of a biocompatible material that is also referred to as a “biomaterial”. The patch may be secured to a portion of the outer periphery of the stent by gluing, stitching, adhering, stapling, suturing, or other means, as described in greater detail hereinbelow.

The cover of the present invention can be provided in tubular form. As an example, the stent cover can be configured as a seamless tubing. The cover of the present invention can be provided in tubular form. In one embodiment, a cover in tubular form may generally be made of a sheet material by joining at the seam where two edges are sewn, coupled, secured, or welded together to form a tubular configuration. In another embodiment, a cover in tubular form can be made of a seamless material such as a blood vessel or an extruded hollow tubing (for example, collagen tubing) where there is no seam around the tubular device. The prosthesis of the present invention may comprise a tubular stent having an outer periphery, with the cover provided about the outer periphery of the stent.

The stent according to the present invention can be any stent, including a self-expanding stent, or a stent that is radially expandable by inflating a balloon or expanded by an expansion member, or a stent that is expanded by the use of radio frequency which provides heat to cause the stent to change its size. The stent can also be made of any desired material, including a metallic material, a metal alloy (e.g., nickel-titanium), a shape-memory material, or even polymeric composites. The stent can have any wire or cell design. Examples of self-expanding wire mesh stents that can be used include the coronary Wallstent™ marketed by Schneider, and the SciMED Radius™ stent marketed by Boston Scientific Corp. Examples of balloon expandable stents that can be used include the Multilink™ stent by Guidant Corp., the Coronary Stent S670 by Medtronic AVE, the Nir™ stent by Boston Scientific Corp., the Cross Flex™ stent by Cordis, the PAS™ stent by Progressive Angioplasty Systems Inc., the V-Flex Plus™ stent by Cook, Inc., and the Palmaz-Schatz™ Crown and Spiral stents by Cordis, among others. The vessels in which the stent of the present invention can be deployed include but are not limited to natural body vessels such as ducts, arteries, trachea, veins, intestines, bile ducts, ureters and the esophagus.

The term “drug” as used herein is intended to mean any compound which has a desired pharmacologic effect. The drug should be compatible with the tissue and can be tolerated in a patient. For example, the drug can be an anticoagulant, such as an RGD peptide-containing compound, heparin, antithrombin compounds, platelet receptor antagonists, anti-thrombin antibodies, anti-platelet receptor antibodies, aspirin, prostaglandin inhibitors, platelet inhibitors, or tick anti-platelet peptide. The drug can also be a promoter of vascular cell growth, such as a growth factor receptor antagonist, transcriptional activator or translational promoter. Alternatively, the drug can be an inhibitor of vascular cell growth, such as a growth factor inhibitor, growth factor receptor antagonists, transcriptional repressor or translational repressor, antisense DNA, antisense RNA, replication inhibitor, inhibitory antibodies, antibodies directed against growth factors, and bifunctional molecules. The drug can also be a cholesterol-lowering agent, a vasodilating agent, and agents which interfere with endogenous vasoactive mechanisms. Other examples of drugs can include anti-inflammatory agents, anti-platelet or fibrinolytic agents, anti-neoplastic agents, antiallergic agents, anti-rejection agents, anti-microbial or anti-bacterial or anti-viral agents, hormones, vasoactive substances, anti-invasive factors, anti-cancer drugs, antibodies and lymphokines, anti-angiogenic agents, radioactive agents and gene therapy drugs, among others. The drug may be loaded as in its/their original commercial form, or together with polymer or protein carriers, to achieve delayed and consistent release.

Specific non-limiting examples of some drugs that fall under the above categories include paclitaxel, docetaxel and derivatives, epothilones, nitric oxide release agents, heparin, aspirin, coumadin, PPACK, hirudin, polypeptide from angiostatin and endostatin, methotrexate, 5-fluorouracil, estradiol, P-selectin Glycoprotein ligand-1 chimera, abciximab, exochelin, eleutherobin and sarcodictyin, fludarabine, sirolimus, tranilast, VEGF, transforming growth factor (TGF)-beta, Insulin-like growth factor (IGF), platelet derived growth factor (PDGF), fibroblast growth factor (FGF), RGD peptide, beta or gamma ray emitter (radioactive) agents.

The cover can be made from either a tissue, a hydrogel, protein matrix, or a polymer, as these terms are defined hereinbelow. The tissues and hydrogels according to the present invention should have high water content and be able to absorb fluids (i.e., liquid drugs, or drugs carried in fluids). The cover may mean a tubular cover that surrounds an outer periphery of a stent. A tubular cover of the present invention can be seamless (as this term is defined herein) and configured for maintaining the stretchably distensible pressure essentially uniform over the circumference of the cover.

The term “tissue” as used herein is intended to mean any mammalian (human or animal) tissue that has sufficient strength and elasticity to act as the primary component of the prosthesis. Tissue should have a cellular matrix of proteins (e.g., collagen). Tissue can include tissue that is obtained from the host patient in which the prosthesis is to be implanted (known as autologous tissue). Tissue can also include homologous tissue, such as from cadavers, umbilical cords, and placenta. In addition, tissue can include heterologous tissue, such as from swine, canine, sheep, horse, etc. Tissue can also include tissue produced in vitro using cell culture methods. In one embodiment of the present invention, luminal tissues (e.g., venous tissue such as saphenous veins, antecubital vein, cephalic vein, omental vein, mesentric vein) are preferred. The tissue can be chemically cross-linked (e.g., by glutaraldehyde, polyepoxy, PEG, UV, etc.) or not chemically cross-linked (e.g., fresh, frozen or cryopreserved). The tissue can also be chemically modified with proper charge and hydrophilicity. The tissue can be harvested according to known techniques, such as those described in Love, Autologous Tissue Heart Valves, R. G. Landes Co., Austin, Tex., 1993, Chapter 8.

Tissue as defined herein can even include tissue that has been processed under the techniques described in U.S. Pat. Nos. 6,106,555 and 6,231,614, whose complete disclosures are incorporated by this reference as though set forth fully herein. These patents disclose a chemical treatment method for tissue fixation and modification by using an epoxy compound. The epoxy compound has a hydrocarbon backbone that is devoid of either an ether or ester linkage. The epoxy compound can also be water-soluble. Thus, tissue may comprise cross-linked tissue or a vein, as these are disclosed in U.S. Pat. Nos. 6,106,555 and 6,231,614. Depending on the embodiment, a vein of porcine, bovine or other mammal origin procured from a slaughterhouse may be chemically treated and used as a stent cover of the present invention. The porcine or bovine vein can be derived from an abdominal region. The mammal vein is particularly applicable because of its stretchability. The stretchability of the stent cover is preferably in the range of 1 ½ to 6 times and beyond. More preferably, the stretchability is in the range of at least twice of its original circumference or original diameter. A tubular cover that is made of a chemical modified seamless tissue using an epoxy compound devoid of either an ether or ester linkage is particularly well adapted for use as a stent cover of the present invention.

Tissue as defined herein can even include tissue that is described in application Ser. No. 09/755,818, filed Jan. 15, 2001 by the present inventor and entitled Vascular Tissue Composition, whose complete disclosure is incorporated by this reference as though set forth fully herein. application Ser. No. 09/755,818 discloses a tissue composition comprising a subendothelial layer, an elastica internal and at least a portion of a tunica media of a blood vessel harvested from a mammal. The tissue composition comprising a subendothelial layer, an elastica internal and at least a portion of a tunica media of a blood vessel may also be chemically treated, such as with glutaraldehyde, formaldehyde, dialdehyde starch, or by the epoxy compound disclosed in U.S. Pat. Nos. 6,106,555 and 6,231,614.

The modified tissue composition as disclosed in patent application Ser. No. 09/755,818 is particularly suitable as a stent cover of the present invention because it retains adequate strength to be mounted on an expanded stent while the modified tissue composition provides improved stretchability and lower profile as part of the tunica and/or adventitial layer is removed in the modified tissue composition. Further, the modified tissue composition may be chemically modified by epoxy compound as disclosed in U.S. Pat. Nos. 6,106,555 and 6,231,614, so as to yield a biocompatible stent cover that maintains essentially most of the original tissue compliance and strength.

The term “hydrogel” as used herein is intended to mean a natural gel-like material that is formed by protein. The hydrogel material has a proper hydrophilicity to regulate the water and drug diffusion process. The release of the drugs is accomplished by other charged particles in the patient's body which competes with the charged binding site in the hydrogel material for the drug. Hydrogel can include albumin, collagen, gelatin, starch, celluloses, dextran, polymalic acid, polyamino acids and their co-polymers or lightly cross-linked forms. Other possible materials are polysaccharides and their derivatives. Yet other possible materials include sodium alginate, karaya gum, gelatin, guar gum, agar, algin, carrageenans, pectin, locust bean gums, xanthan, starch-based gums, hydroxyalkyl and ethyl ethers of cellulose, sodium carboxymethylcellulose. Some are food gels and some are bioadhesives.

The term “material” as used herein means either tissue, hydrogel or the like.

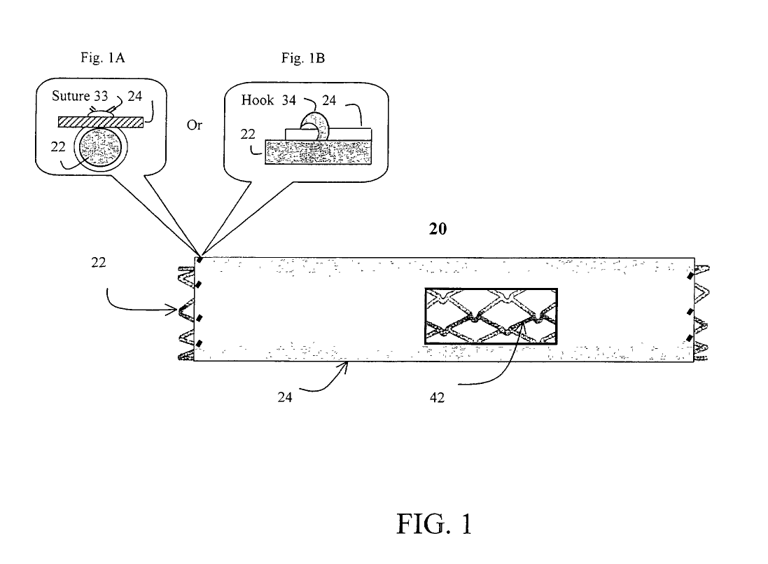

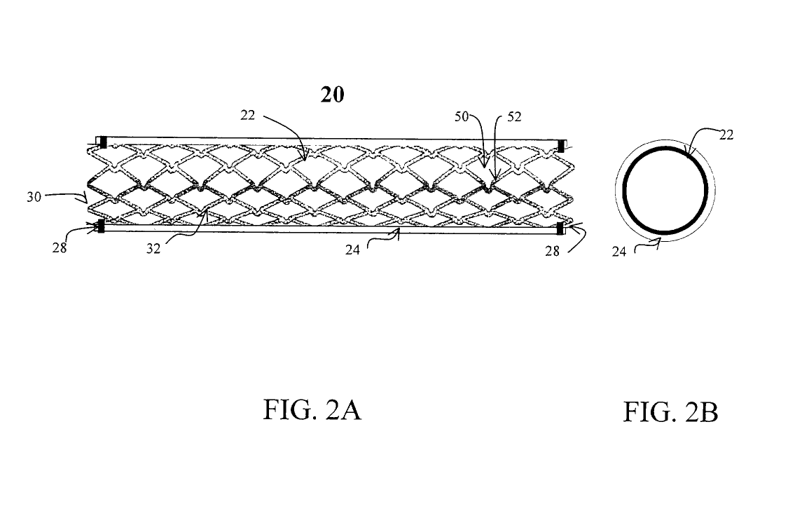

FIGS. 1, 2A and 2B illustrate a prosthesis 20 according to one embodiment of the present invention. The prosthesis 20 has a tubular stent 22 and a cover 24 attached over the outer periphery of the stent 22. As described above, the stent 22 can be any known or conventional stent, and as a non-limiting example, FIG. 2A illustrates the stent 22 as being a balloon-expanding Nir™ stent by Boston Scientific Corp., as described in FIG. 8 of U.S. Pat. No. 5,733,303 to Israel et al., whose disclosure is incorporated herein as though fully set forth herein.

The stent 22 in FIG. 2A comprises a plurality of flexible cells 50 having a patterned shape, with each cell 50 being formed by a plurality of wall members or struts 52. Each cell 50 is interconnected to a plurality of other adjacent cells to define the patterned shape. These struts 52 can be configured to be either parallel, or perpendicular to, or disposed at an angle with respect to, a longitudinal axis of the tubular stent 22. Each circumferential row of struts 52 can also form a zig-zag cylindrical element that is independently expandable in a radial direction, with the cylindrical elements being interconnected and configured so as to be generally aligned on a common longitudinal axis. In another embodiment, the tubular stent can have a plurality of diagonal elements connected to a longitudinal frame and configured to exhibit an undulating contour for enhancing longitudinal flexibility. Alternatively, the tubular stent may comprise a plurality of cylindrical elements that are independently expandable in a radial direction, the cylindrical elements being interconnected and configured so as to be generally aligned along a common longitudinal axis.

The cover 24 acts as a drug reservoir that stores the drug(s) to be released at the site of implantation of the prosthesis 20. The cover 24 is extensible (i.e., can be stretched) and flexible, and has the ability to absorb drugs and to store the drug(s) before the prosthesis 20 is deployed. The cover 24 can be either a single-layer of material (such as tissue or hydrogel) or multiple layers of material. When multiple layers are used, the layers can include (1) tissue with hydrogel layer, (2) polymer (non-drug loading) layer with hydrogel layer, (3) polymer (non-drug loading) layer with cultured tissue layer (e.g., culture collagen, elastin, crosslinked soluble protein, etc.), (4) hydrogel layer with hydrogel layer (e.g., two hydrogel layers having different drug release rates), and (5) polymer (non-drug loading) layer with tissue layer, among others. In the multiple-layer configuration, at least one material layer will absorb the drug, and one of the layers can be a non-drug loading layer. The non-drug loading layer would not contain any drug(s), and may be made of nonhydrogel polymers, such as polyurethanes, expanded PTFE, polyesters, polyamides, polylactide, polylactide-co-glycolide, polydioxanone, thermoplastic elastomers, thermoplastics, silicone rubbers, or other polymers. The non-drug loading layer facilitates directional drug delivery since this layer forms a barrier against drug diffusion.

In the embodiment of FIGS. 1-2B, there are a number of ways of loading the drug(s) to the cover 24. The material utilized for the cover 24 may have water content greater than 90% by weight. If so, the water can be removed by a lyophilization process that is a well-known technique in the art. If tissue is used for the cover, the drug(s) can be loaded onto the tissue material by impregnation, soaking, coating, adsorption or absorption, even if the tissue material has been processed using the epoxy compound described in U.S. Pat. Nos. 6,106,555 and 6,231,614.

One method involves physical absorption into the cover 24. Under this method, the drug is loaded into the material during the rehydration process. The drug may be dissolved in a physiological solution for rehydration of the lyophilized material. If the drug has limited solubility in water, additional solvent may be added to facilitate the dissolving process, as long as the solvent has no adverse effects on the cover and the host patient. As an example, ethanol at a concentration of less than 50% v/v may be suitable for the rehydration process. The rehydration process for tissue and hydrogel is fast, easy and complete. The material has no noticeable change in property before dehydration and after complete rehydration. By changing the hydrophilicity of the material, the drug may be released at different rates.

A second method involves the use of a charged chemical to electronically attract and retain drugs. In particular, natural tissue and the hydrogels defined above are proteins, which are composed of amino acids with various kinds of functional groups. By choosing the appropriate modification reagent, it is possible to selectively reduce certain groups to imbalance the surface and matrix charge of the tissue or hydrogel to either positive or negative. For example, aldehyde group will react with amino group to change the surface and matrix charge to negative. Carbodiimide reaction will target the free carboxyl group to change the surface and matrix charge to positive. Addition of charged chemicals into tissue may also change the net electricity of the tissue. A charged tissue or hydrogel material has the tendency to electronically attract and retain a drug carrying the opposite charge. The drug will then be released inside the vessel after implantation. The release of the drugs is accomplished by other charged particles in the patient's body which competes with the charged binding site in the hydrogel material for the drug.

A third method involves chemical reaction or bonding to link certain drugs to the material. The bonding may be covalent or ionic. For example, heparin may be immobilized to tissue surface covalently through direct carbodiimide reaction or with polyethylene oxide as a bridge or spacer. Heparin can also link to tissue through ionic interaction through benzalkonium or stearylkonium. The drug may be released or remain on the surface of the tissue or hydrogel with activity in the vessel.

A fourth method involves coating the surface of the tissue or hydrogel. For example, the drug can be sprayed onto the surface, and then a gel-like material may be used to coat the tissue or hydrogel. As another example, it is also possible to first mix the gel with the drug, and then coat the mixture on to the material. As yet another example, the gel may be applied over the outer layer of the tissue or hydrogel before the drug is loaded. Then, just before implantation, the cover 24 can be immersed in a solution containing the drug, and the nature of the gel will cause the drug to be retained or loaded in the gel. The prosthesis 20 can then be delivered inside the desired vessel and the drug will be released over a period of time. Examples of the gel-like material may include polyethylene oxide, polyvinyl pyrrolidone, polyacrylates, and their blends or co-polymers or lightly crosslinked forms. Other examples include polyethylene glycol block copolymers with polylactides or other polyesters. Yet other examples include hydrophilic polyurethane, poly(maleic anhydride-alt-ethylene) and their derivatives. Further examples include polysaccharides and their derivatives, sodium alginate, karaya gum, gelatin, guar gum, agar, algin, carrageenans, pectin, locust bean gums, xanthan, starch-based gums, hydroxyalkyl and ethyl ethers of cellulose, sodium carboxymethylcellulose. Some of these gel-like materials can be heated and then cooled to form the gel. Some are food gels and some are bioadhesives.

Referring to FIGS. 1 and 2A, the cover 24 can be attached to the stent 22 by suturing the ends 28 of the cover 24 to the desired portions of the stent 22. For example, the cover 24 can be about the same length as the stent 22, in which the ends 28 of the cover 24 are sutured (e.g., see suture 33 in FIG. 1A) to the ends 30 of the stent 22. If the length of the cover 24 is less than the length of the stent 22, then the ends 28 of the cover 24 can be sutured to selected wires (e.g., 32) of the stent 22 so that the cover 24 covers a portion of the stent 22. Other methods of attachment include the use of hooks or barbed mechanisms 34 on the stent 22 to hook the cover 24 to the stent 22 (see FIG. 1B), or the use of glue to attach selected portions of the cover 24 to selected portions of the stent 22. Another method of attachment can include the use of an overlaying or wrapping membrane that covers the cover 24 and the stent 22, but which is removable with the delivery catheter after the prosthesis 20 has been delivered to the desired location in the vessel.

The cover 24 can be provided in the form of a tubular cover (i.e., luminal) or as a sheet that can be formed into a tubular cover by suturing or stitching side edges of the sheet. If the cover 24 is luminal, the cover 24 can be slid over the stent 22 and then attached. If the cover 24 is provided in the form of a sheet of material, the sheet of material can be merely wrapped around the stent 22, and no stitching is required. In either case, the attachment can be done with the stent 22 in the expanded state or in the compressed state. If the attachment is done in the expanded state, the prosthesis 20 is then compressed to a smaller diameter for delivery. When the prosthesis 20 is compressed, the flexible and stretchable nature of the cover 24 would allow the cover 24 to compress with the stent 22 without any creasing. Similarly, if the attachment is done in the compressed state, the flexible and stretchable nature of the cover 24 would allow the cover 24 to expand (e.g., stretch) with the expanding stent 22 when the prosthesis 20 is expanded.

The cover 24 can have at least one perforation 42 as shown in FIG. 1. The perforation 42 is typically positioned at the location of a branched blood vessel.

The cover 24 is made from a material that is devoid of any live endothelial cells. The endothelial cells are lined at the blood contact surface of a blood vessel with about one-cell thickness, and function to render the blood contact surface hemocompatible and to provide a certain degree of immunogenicity to fend off foreign substances. To use a tissue material as the stent cover 24, it is desirable to remove or to kill (e.g., by cross-linking the tissue) all of the endothelial cells so as to reduce any undesired immunogenicity.

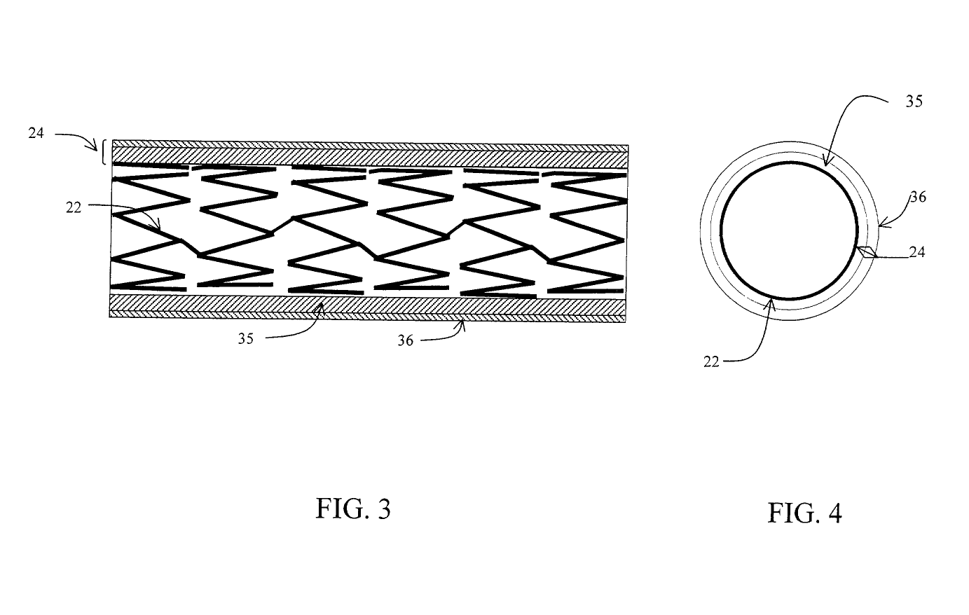

The cover 24 can comprise two layers of material, with at least one layer made from a water-absorbent material. An inner layer of the two layers of material may be configured for contacting the outer periphery of the tubular stent. The inner layer can be impermeable to the delivery of drugs so that no drug therapeutic effects are introduced into the lumen of the prosthesis. Alternatively, the outer layer of the two layers of material may be an exterior side of the two layers and configured for contacting the tissue of a body conduit. Here, the outer layer can be impermeable to the delivery of drugs so that no drug therapeutic effects are introduced onto the tissue of the body conduit. The two layers are shown in FIGS. 3 and 4, with the inner layer designated by 35 and the outer layer designated by 36.

In one embodiment, at least one layer of of the two layers of material is made from a water-absorbent material. One of the two layers has a material which is selected from a group consisting of a matrix of protein, tissue, hydrogel, polymer, and cultured tissue layer, wherein said polymer may be selected from a group of polyurethanes, expanded PTFE, polyesters, polyamides, polylactide, polylactide-co-glycolide, polydioxanone, thermoplastic elastomers, thermoplastics, and silicone rubbers, and wherein said cultured tissue layer may be selected from a group consisting of cultured collagen, cultured elastin, and crosslinked soluble protein.

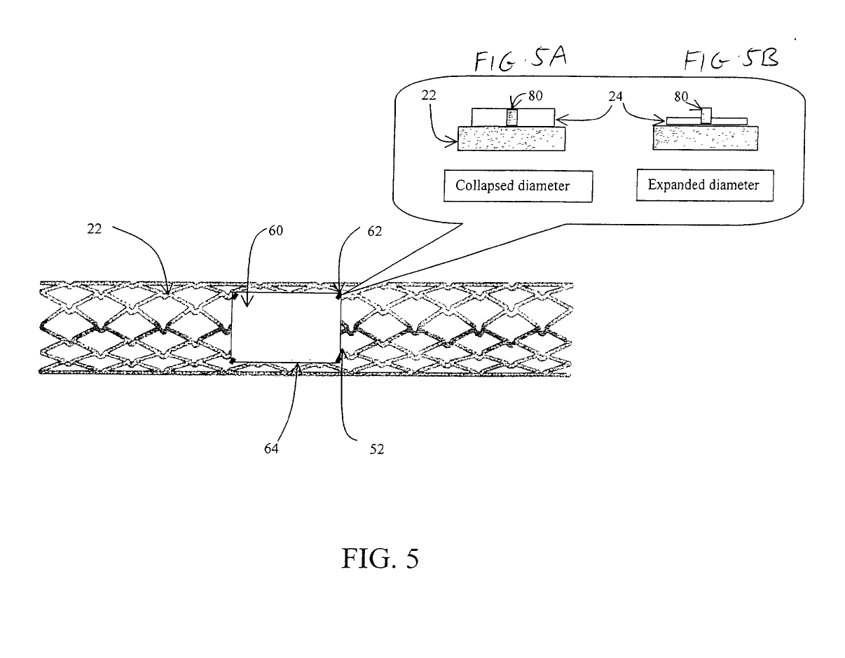

Referring to FIG. 5, the cover is illustrated as being a patch 60 that can be secured to the stent 22 at selected locations. For example, each of the four corners 62 of the patch 60 can be stitched to a separate strut 52. Alternatively, one or more of entire edges 64 of the patch 60 can be secured (e.g., by stitching) to the stent 22. The patch 60 preferably has the same stretchable characteristics as those described above for the cover 24. The patch 60 can be secured to the stent 22 when the stent 22 is in the compressed or expanded state.

The patch 60 can be important when a cover 24 is used to treat vulnerable plaque. Typically a vulnerable plaque site is about 1 mm (0.2 to 2 mm range) in diameter. Therefore, the patch 60 holds the vulnerable plaque site from erosion or rupture. The surrounding tissue adjacent to a vulnerable plaque site does not need any cover.

The prosthesis 20 can be implanted using any known implantation methods for the underlying stent 22. A catheter can be used to deliver the prosthesis 20 to the desired location in the vessel, and then the stent 22 can be expanded (i.e., either self-expanding or balloon expanded, depending on the type of stent). In essence, the prosthesis 20 will be deployed and used in the same manner as its underlying stent 22. The deployment techniques and functions of the stent 22 are well-known, and shall not be explained in greater detail.

The drug contained in the cover 24 can be released by diffusion, or by any of methods described above. Since tissue and hydrogel are water permeable, water and molecules can diffuse through the tissue or hydrogel cover 24 at different rates. The diffusion rate can be controlled by varying the thickness of the cover 24, changing the size of the migrating molecules (either the drug alone or with a carrier to form a larger molecule to slow down the diffusion process), changing the hydrophilicity of the cover 24, changing the drug concentration (i.e., drug released from its polymeric carrier), and coating the surface of the cover 24 with polymeric material having different permeability.

Thus, the cover 24 of the present invention provides a sheath or sleeve to block the path of cell migration (i.e., ingrowth), and to pave or act as a scaffold for supporting the lumen. The cover 24 acts as an effective drug delivery device for locally delivering a drug to an arterial wall or lumen into which the prosthesis 20 has been inserted and positioned.

### EXAMPLE 1

A dried tissue stent cover made of polyepoxy crosslinked porcine venous tissue, 25 μm thick at its collapsed diameter and 30 mm long (0.5 mg dried weight), is soaked in approximately 5 mg of water or any liquid medication during its rehydration process.

### EXAMPLE 2

A polymeric stent cover, made of ePTFE, is provided with another layer of Taxol, gelatin, and poly(e-caprolactone) mixture (20:20:60) on the outside. 20% of the Taxol is released to the artery wall during the first week after implantation.

Vulnerable Plaque

To varying degrees, an atheromatous lesion is comprised of a lipid-rich core, a cap of fibrous tissue, vascular muscle cells expressing collagen and elastin that impart tensile strength to an extracellular matrix, and inflammatory cells that produce various enzymes and procoagulant factors. For illustration purposes in the present invention, an atherosclerotic plaque is generally divided into two categories: a vulnerable plaque and a stable plaque. A stable plaque is generally characterized by the most conspicuous stenoses, that is, the angiographically significant (greater than 70% diameter narrowing) lesions versus a large number of insignificant (less than 50% diameter narrowing) unapparent lesions (called vulnerable plaque).

After angioplasty on a stable plaque, a stent is typically implanted intraluminally. The pressure to deploy a stent by an expandable balloon is generally in the range of 6-10 atmospheres or higher. The stent 22 and its stent cover 24 for use with a stable plaque functions to maintain the lumen dimension and prevent stent restenosis. The stent 22 should have sufficient circumferential strength, but the requirement for longitudinal strength will not be as significant.

On the other hand, a vulnerable plaque will cause little luminal narrowing and is generally not angiographically viewable. The fibrous cap, which is characterized by a single endothelial cell layer, may be thinned and partially eroded by both inflammatory T-lymphocytes and invading smooth muscle cells. Abundant activated macrophages moving into the plaque from the vasa vasorum produce proteolytic enzymes, such as matrix metalloproteinases, that promote collagen degradation, which leads to cap disruption and the thrombogenic surface activation associated with acute coronary syndromes. The cover 24 of the present invention can not only be used to treat the restenosis of a stable plaque, but can also be used to treat/prevent the rupture or erosion of a vulnerable plaque.

An endoluminal cover (such as cover 24 or patch 60) to cover vulnerable plaque can be supported by a supporting element or a low pressure stent, such as stent 22. The supporting element holds the cover 24 or patch 60 against the luminal wall of the vessel to prevent the rupture of the vulnerable plaque. There is little pressure exerted from the supporting element onto the cover 24 or patch 60. The stent 22 used in this application should maintain a similar circumferential force against the luminal wall of the vessel. The vulnerable plaque is a lesion inside the vessel wall in a morphology that the vulnerable plaque does not protrude into the lumen of a blood vessel. Therefore, there is no need to push outwardly or to stent the vulnerable plaque. However, to maintain the stent cover 24 or patch 60 on top of a vulnerable plaque, a supporting element (such as a stent) is needed at least on a temporary basis.

The circumferential force of a stent holds the stent in place at the location of the lesion region against forces such as the flow of blood, and any hemodynamic effects on the stent. Typically the circumferential force of a stent is moderate in either treating a stenosed stable plaque or a vulnerable plaque. The circumferential force to hold a stent in place is believed to be about 5 to 150 mm Hg (absolute), preferably 10-50 mm Hg. It is also noted that the typical diastolic pressure is 80 mm Hg and the systolic pressure is typically 120 mm Hg for a healthy person. Therefore, it is reasonable to assume that the holding force (or pressure) is moderate.

On the contrary, the radial force of a stent used to treat a stable plaque is large since it is necessary to break out the calcified or solidified atherosclerotic plaque, so this radial force is in the 10 to 15 atmosphere pressure range.

The circumferential force for a stent cover 24 that is used for vulnerable plaque should be sufficient to hold the cover 24 and its supporting element in place. Therefore, the circumferential force for a stent cover 24 used for treating vulnerable plaque should be about equal to that of the circumferential force for a stent cover used for treating a stable plaque. If the radial force of a stent cover 24 for vulnerable plaque is too large, then the vessel wall might be pushed outwardly to cause a false aneurysm. As discussed above, the circumferential force to hold a stent in place is about 5 to 150 mm Hg (absolute), and preferably 10-50 mm Hg. In other words, the circumferential force of equal to or less than the systolic pressure (nominally 120 mm Hg) is generally within the safety range for the blood vessel wall.

Another method for holding the stent cover 24 in place for treating vulnerable plaque would be to use a “frictional force” exerted by the exterior surface of the stent cover 24 to the underlying tissue surface. Since a rough surface has higher friction (under the same circumferential force scenario), the stent 22 and its stent cover 24 can be provided with a micro-level rib, protrusion, wavey, studded cover surface on the outer surface of the stent cover 24, which will increase the surface friction between the cover 24 and the contacted tissue. The rib, protrusion (e.g. 80 in FIGS. 5A and 5B), wavy or studded phenomena can be part of the stent 22 itself. The anchoring protrusions 80 may be fully embedded in the stent cover 24 before expansion of the stent 22, and they are exposed and help anchor the patch 60 or cover 24 to the vessel wall when the patch 60 or cover 24 is stretched during expansion of the stent 22. It is also possible to achieve this anchoring effect by employing the stent structure itself. A stent 22 particularly useful for this vulnerable plaque application can have a stent cell size of 0.05 to 0.25 mm during the retracted (i.e., non-deployment) state and subsequently enlarged to a cell size of 1 to 2.5 mm size after expansion (i.e., deployment). The larger cell size renders the outer surface of the cover 24 rougher or uneven for better anchoring onto the tissue surface of a vulnerable plaque.

The stent cover 24 could be loaded with drug effective to prevent, slow-down or even reverse the vulnerable plaque process.

The cells 50 of the stent 22 should be small enough to yield a uniform force distribution on the luminal wall of the vessel circumferentially. The average cell size for the stent 22 (to be positioned at an area having vulnerable plaque) is preferably less than 3 mm. Similarly, the maximum wire to wire distance (or the equivalent diameter of the cell area as defined as the circumference divided by π) within a stent 22 should not be more than 3 mm. The covers 24 used for treating vulnerable plaque may be less stretchable as compared to the covers 24 used for treating stable plaque because the stent 22 used for vulnerable plaque applications will be less expandable, since a vulnerable plaque does not need to be expanded (a vulnerable plaque has no stenosis). On the other hand, a stent 22 used for treating a stable plaque needs to expand a little more so as to stent the stable plaque back to the nominal lumen diameter because a stable plaque has a stenosis and the plaque protrudes into the vessel lumen. Thus, when used to treat vulnerable plaque, the cover 24 functions to provide a scaffold for containing the vulnerable plaque from rupture or erosion. Additional steps to further secure the cover 24 to the vulnerable plaque area may include applying adhesives, local polymerization, and physical energy (e.g., laser, heat) to fuse the cover 24 to the wall of the vessel.

The present invention also provides a method for treating a vulnerable plaque. According to this method, a drug can be loaded into a cover 24 or a patch 60 using the techniques described above, and the cover 24 or patch 60 is delivered to the location of the vulnerable plaque. The cover 24 or patch 60 is internally supported by a supporting element, which may comprise an expandable stent 22. The stent 22 and the cover 24 (or patch 60) are implanted over the vulnerable plaque by expanding the stent 22. The cover 24 can surround an outer periphery of the stent 22, and can comprise at least one layer of material, or even two layers of materials, as described above. In particular, the cover 24 surrounds at least a portion of, or the complete length of, an outer periphery of the stent 22. A perforation (similar to 42) can be provided in the cover 24 or patch 60. The cover 24 or the patch 60 is sized and configured to adequately cover the entire vulnerable plaque.

As an alternative, the cover 24 can surround at least a portion of both an inner periphery and an outer periphery of the stent 22. The cover 24 can be configured to have a continuous coverage at an end or both ends of the stent 22.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A method for treating a vulnerable plaque, the method comprising implanting a prosthesis over the vulnerable plaque, wherein the prosthesis comprises a tubular stent having an outer periphery and a cover provided about the outer periphery of the stent, the cover made from a water-absorbent material.

2. A method for treating a vulnerable plaque, the method comprising implanting a prosthesis over the vulnerable plaque, wherein the prosthesis comprises a stent and a cover surrounding at least a portion of both an inner periphery and an outer periphery of the stent.

3. A method for treating a vulnerable plaque, the method comprising delivering a drug to the vulnerable plaque, wherein the drug is loaded into a cover that is secured against the vulnerable plaque.
